(19) United States
(12) Patent Application Publication
(10) Pub. No.: US 2018/0146644 A1
(43) Pub. Date: May 31, 2018

(54) METHOD OF MEASURING REACTION TIME IN ANIMALS

(71) Applicant: Vium, Inc., San Mateo, CA (US)

(72) Inventors: Jonathan Betts-Lacroix, Belmont, CA (US); Laura Schaevitz, Los Gatos, CA (US)

(73) Assignee: Vium, Inc., San Mateo, CA (US)

(21) Appl. No.: 15/364,110

(22) Filed: Nov. 29, 2016

(51) Int. Cl. A01K 29/00; A61B 5/16; G01G 23/37

(52) U.S. Cl. CPC A01K 29/005; A61B 5/162; A61B 2562/0219; A61B 2503/40; G01G 23/3728

(57) ABSTRACT

Methods of automatically measuring a reaction time of an animal, in a cage, to a stimulus, are described. First, an animal is detected, then a second apparatus waits for the animal to be fully on the apparatus. A first apparatus outside the cage then provides a stimulus. A second, sterilizable, wireless apparatus inside the cage measures a response of the animal such a jerk, using an accelerometer. The first and second apparatuses time-synchronize. The second device transmits wirelessly the response time data, after optional preprocessing, to the first apparatus. For multi-housed animals, an ID of the animal is determined. The second apparatus may comprise an animal weight scale.

### BACKGROUND OF THE INVENTION

Prior art methods of determining the reaction time of a rodent remove the animal from its home cage, arrest it in a specially equipped cage, make a loud sound, then observer the animal jump.

Weakness of this method are: inaccurate measurements; inconsistent measurements; stressing the animal because it is in a foreign environment; measurements are made during daytime, not during the animal's nocturnal activity period. the number of measurements is limited; inability to track changes in reaction time; inability to use measurements to determine or predict progress of a disease or treatment.

### SUMMARY OF THE INVENTION

Embodiments of this invention overcome the weaknesses of prior art.

Embodiments of this invention use a sterile, wireless second apparatus in the animal's home cage to automatically measure an animal's reaction time. The cage or apparatus includes one or more stimuli sources, such as sound, light or motion. The apparatus includes an accelerometer and wireless communication. The cage or apparatus includes animal location detection equipment so that reaction time is measured only when the single animal of interest is fully on the apparatus.

In some embodiments, a stimulus is provided by a first apparatus, associated with the cage. For these embodiments the first apparatus and the second apparatuses have a synchronization step to assure that the time from the stimulus to the animal's response is accurately measured.

In some embodiments, the second apparatus also includes a sterile, wireless weight scale.

In some embodiments the first apparatus also includes a camera used to uniquely identify an animal on the first apparatus in a multi-housed cage.

In some embodiments multiple reaction time measurements are taken sequentially and the sequence is compared to a disease model to measure disease progress, predict future disease state, or measure treatments success.

### BRIEF DESCRIPTION OF THE DRAWINGS

FIG. 1 shows a time line of one embodiment, including stimulus, response and communication.

FIG. 2 shows an overhead view of a cage equipped with a detection and communication apparatus.

FIG. 3 shows a side view of a cage with a pair of apparatuses for stimulus, measurement and communication.

FIG. 4 shows a side view of a detection apparatus.

FIG. 5 shows a flow chart of one embodiment.

FIG. 6 shows exemplary raw data and preprocessed data for a response.

FIG. 7 shows times embodiments for the timing of animal ID.

### DETAILED DESCRIPTION

Scenarios and options are non-limiting embodiments.

The essence of reaction time is to provide a stimulus and measure a response. A response time is the elapsed time from the stimulus to the response.

A stimulus may be a sound, such as a click, tone, burst of white noise, a bell, or any of many other choices. One common choice is a 50 ms burst of white noise at 100 db. A stimulus may be light, such as a flash. Many choices exist, including different colors, different directions, and may include turning off a light, instead of turning on a light. A stimulus may be a motion, such as a jerk or vibration. A stimulus may be an electric shock. A stimulus may be sudden heat or cold. Stimulus may be a burst of air. Stimulus should be selected for consistent response, in otherwise the same conditions. For this reason, a loud noise is often used. In a vivarium, it is strongly desirable that the stimulus in one cage not “leak” into other cages. Providing an excess or unnecessary stimuli may cause habituation or otherwise alter results of a study. Stimulus may be provided by a speaker, a piezoelectric transducer (PZT), a light source such as a flash lamp or LED, or other source.

An animal's reaction time response is typically a muscle jerk. This may be detected by an accelerometer, if the animal is connected motionally to the accelerometer. For example, an animal may be on the top of an apparatus in the cage with an accelerometer functionally connected to the top of the apparatus. There are other ways to detect a response. For example, video at a suitably fast frame rate and suitably fast resolution may be used. A healthy mouse reaction time is about 35-40 milliseconds (ms). Therefore, a suitable video frame rate is at least 60 frames per second or higher, in order to measure variations in reaction time.

In one embodiment there are two apparatuses. A first apparatus is dedicated to one cage, and may be located above the cage, for example. The second apparatus is located in the cage with the animal. This apparatus is sterilizable, as is required (unless disposable) for use in animal studies. The second apparatus is wireless and there are no electronic penetrations of the cage. Therefore, the second apparatus needs a battery or other internal power source. The second apparatus detects the response of the animal to the stimulus. The second apparatus communicates this detection to the first apparatus. Ideally, optical communication is used; ideally this communication is bi-directional, although not all embodiments require bi-directional communication.

Stimulus may be provide by the first apparatus, the second apparatus, or by another source. If the second apparatus provides the stimulus then response time measurement is relatively simple as the electronics for both the stimulus and response are co-located in the second apparatus. Stimulus may be provided by the first apparatus, which has several advantages. First, the power needed for the stimulus does not drain the power source of the second apparatus. Second, the first apparatus may have a better overview of the cage and thus may be able to provide a more uniform stimulus. Third, stimulus from the first apparatus may be lower cost and easier to maintain. Since the second apparatus must be sealed with respect to pathogens, it may be easier, lower cost and more effective to provide the stimulus from the first apparatus, since it is outside the cage and thus has fewer sealing requirements.

However, if the stimulus is provide by the first apparatus outside the cage and the response is measured inside the cage by the second apparatus, there must be a synchronization event between the two apparatuses. One such method is for the second apparatus to detect the stimulus, such a by an audio receiver for an audio stimulus or a light sensor for a light stimulus. In this arrangement, no other synchronization is needed as the stimulus itself also provides the necessary time synchronization. An alternative is to use another method of synchronization, which may be provided using the communications link, whether it is bi-directional or unidirectional. A synchronization signal may be sent in either direction.

Synchronization may occur before a stimulus-response, during, or after. If the synchronization occurs after then the necessary arithmetic must be performed to compute a proper response time.

In some embodiments the second apparatus is also a scale. It may communicate weight to the first apparatus, using the wireless communication elements.

In some embodiments, the second apparatus communicates with the first apparatus using optical communication, such as from the top of the apparatus, through a transparent cage top, to the first apparatus.

In some embodiments, this optical communication is redundant, at least on the second apparatus end, so that if an animal is obscuring one transceiver, the second one is likely no obscured. Ideally, the two (or more) optical transceivers are spaced on the top of the second apparatus such that a typical animal is not able or unlikely to cover both.

Sharing the functions of an animal weight scale with an animal response time detector is particularly efficient and economical, as most of the electronics for the two functions may be shared, such as the sealed case, power source, processor, and communications. In addition, a cage may not be large enough for two separate devices.

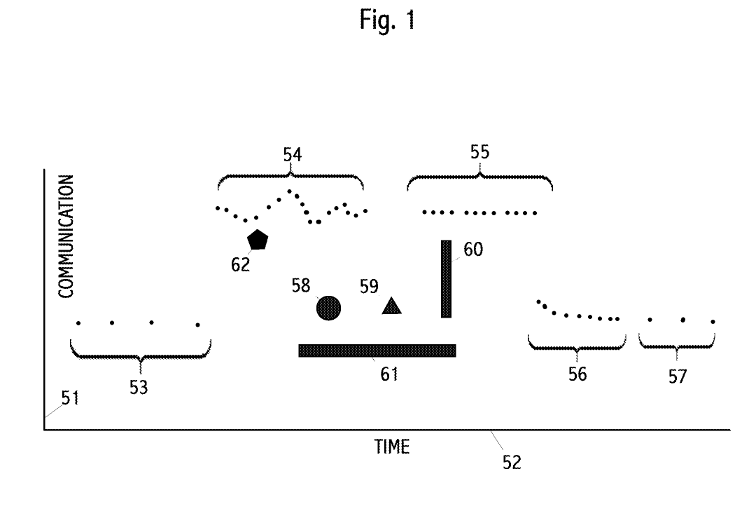

Turing now to FIG. 1, we see a number of embodiments. The horizontal axis 52 is a time line. The vertical axis 51 is not a metric; it provides convenient space in the Figure to show events. Each small dot, such as shown within the brackets, 53, 54, 55, 56, and 57 may represent one block, packet or frame of communication between the first and second apparatuses. We see in bracket 53 that a low data rate is being used because there is no animal activity for the second apparatus to report. At the start of 54 an animal is on the second apparatus. The data rate increases, as the second apparatus, for example, communicates changing animal weight due to animal motion. Event 62 is a command from the first apparatus to the second apparatus to detect a reaction time. This command may occur any time prior to the actual stimulus 58. It may be days earlier, or an implied command as part of some other communication or a configuration. Circle 58 is the stimulus event. The stimulus may be generated from either the first or second apparatus. 59 is the animal's response to the stimulus 58. The response is detected by the second apparatus, such as by an accelerometer. The time from 58 to 59 is the animal's reaction time. At event 60, the second apparatus starts communicating the time measured at 59 to the first apparatus. The dots in bracket 55 show schematically how such information may be a series of data transmissions from the second apparatus to the first apparatus.

As discussed elsewhere herein, it is necessary that the first and second apparatuses be time synchronized if the stimulus is from the first apparatus. This is shown schematically as bar 61. Note that synchronization may occur at almost any time, including before, during or after the stimulus 58 and the animal's response 59. It may occur after communication 60, noting that correction may be required so that the final recorded animal's response time is responsive to any synchronization time correction.

Communication dots in 56 show schematically communication after response time communication is complete. This may be, for example, additional animal weight transmissions, error detection, status data, or other information. Communication dots in bracket 57 show the data rate from the second apparatus to the first apparatus dropping back to a low, background data rate. Changes in data rage are not required for all embodiments. However, they are advantageous to conserver power in the second apparatus. Background data rate may be one burst every 1 to 60 seconds. Active data rage may be in the range of 1 Hz to 10 KHz. Data rates may be significantly different than these ranges. Data rates may refer either to a packets/second metric or to a bits/second metric.

Embodiments are fully automatic in that no human activity is required for events, 58, 59, 60 and 61. Ideally, every element shown in this Figure is automatic.

Embodiments implement all elements shown in this Figure independently for each cage in an environment, such as a vivarium, with multiple cages.

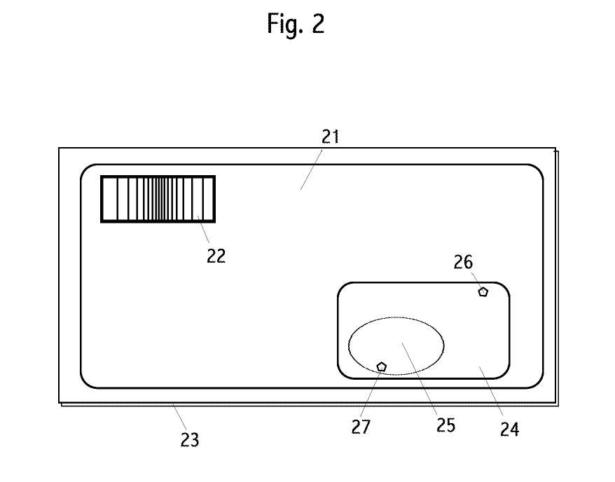

Turning now to FIG. 2, we see a schematic top view of a cage with a first and second apparatus. This Figure is highly schematic and not to scale. 21 is the interior of the cage. 23 shows the outline of a first apparatus, here, slightly larger than the cage, such as sitting on top of the cage. Embodiments do not require any particular arrangement, location or size of the two apparatuses. 24 shows the second apparatus, here located in a corner of the cage, 21. A mouse is shown 25, on the second apparatus. Item 22 is schematically any other element in the cage, such as an exercise device, hutch, water or feeder. It is shown in this Figure to emphasize that embodiments keep the animal in its natural home cage.

Two redundant communication elements are shown, 26 and 27, that optically communicate from the top of the second apparatus 24 to the first apparatus, 23. Note that the mouse 25 is obscuring transceiver 27 but not 26, due their spacing being larger than the mouse's average diameter. The first apparatus may have either a single or redundant communication transceivers.

Embodiments are not limited to optical communication and are not limited to communication elements in the top of the second apparatus. However, there are unique benefits to this configuration, including the ability to implement a large number of closely spaced cages in a vivarium without the problems of radio interference and radio ID pairing.

It is important that the second apparatus 24 is sterilizable to maintain the viability of animal studies in a vivarium and thus it must be sealed against pathogens. Also, the cage 21 should have no electrical penetrations as they are likely to compromise required cage sterility and also animals will chew on everything they can, including electrical wires and most plastic and rubber.

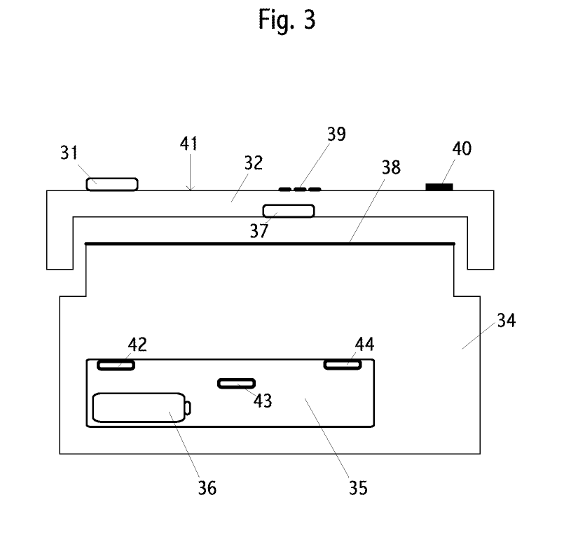

Turning now to FIG. 3, we see a schematic side view of embodiments comprising a first and second apparatus and communication transceivers. 34 is a cage with a transparent top 38. 32 is an electronics “slab” on top of the cage. This slab 32 is the first apparatus or comprises a first apparatus. 35 is the second apparatus, which comprises a power source 36. 43 is a motion sensor to detect animal response, such as an accelerometer, load-cell, optical sensor, capacitive sensor, or other location, motion or acceleration sensor. An animal, not shown would be on top of the second apparatus, 35, prior to stimulus. Ideally the animal is fully on top. The second apparatus may also comprise a weight scale to weigh the animal, not shown. Such a weight scale may be used to detect an animal on top and to detect more than one animal on top and to detect that an animal is fully on top. Such capabilities are explicitly claimed as embodiments. Such detection may be done by starting with a known, expected, average or nominal animal body weight, then bounding that weight with a tolerance, such as ±1%, 2%, 3%, 5%, 7%, 10%, 12%, 15%, 20%, 25%, 30%, 40% or 50%. Such bounding tolerance may be asymmetric, such as minus 5% and plus 10%. Such detection may include averaging and a minimum time within the selected tolerance bounds. such as ½, 1, 1.5, 2, 2.5, 3, 4, or 5 seconds.

42 and 44 are redundant communication transceivers, which may communicate unidirectional or bi-directional. They may be optical or radio. If optical, they may be in an IR band, visible light, UV light, or another band. A specifically claimed embodiment is directionality towards an associated transceiver on the first apparatus such that the light does interfere with camera operation from a camera observing the cage interior. A specifically claimed embodiment is an optical spectral filter such that the light does interfere with camera operation from a camera observing the cage interior. A preferred embodiment is to place the two or more transceivers 42 and 44 at a distance apart such than an animal on the second apparatus 35 is unable or unlikely to block both at the same time. For example, they may be place farther apart than the average or maximum body diameter or animal length (excluding tail) of an animal.

37 is an associated transceiver in the first apparatus. Only one is shown, however, this transceiver may be implemented redundantly, particularly if the optical beam width from 42 and 44 is narrow and vertical.

31 is an optional camera in the first apparatus that observes all or a portion of the cage interior. One use of the camera is to ID an animal in a multi-housed cage. 39 is an optional process or non-transitive memory. 41 may be a circuit board (PCB) on or in the first apparatus to comprise some or all of electronic elements of embodiments. 40 is an optional communication device, such as an electronic network transceiver, either wired or wireless. Ideally, multiple cages in a vivarium are networked via devices 40. Embodiments use the same camera 31 to observe animal behaviors in the cage during the nighttime period of the animal. Embodiments do this observing using IR light.

In one embodiment cages 34 slide on rails in and out of vivarium racks, while slabs 32 remain in the rack. Ideally, there are no electrical penetrations through the walls of the cage 34 and no wired connections, including no electrical connectors, between the first apparatus 32 and the second apparatus 35. However, embodiments wherein the first apparatus is fully or partially in the cage are claimed.

FIG. 3 is not to scale. Shown element shapes are not representative of actual shapes or sizes of elements.

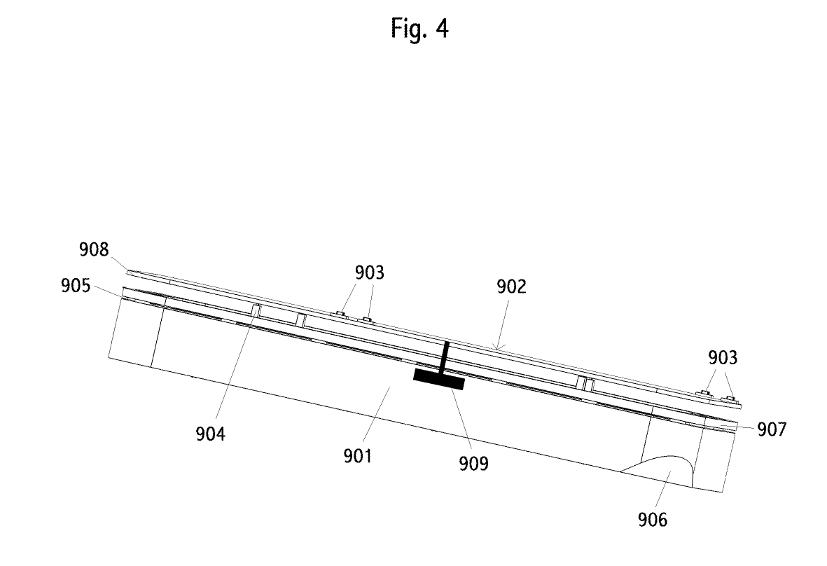

Turning now to FIG. 4 we see a side view of an embodiment of a second apparatus. 901 is the apparatus. 906 is a corner shape adapted so the apparatus fits in a cage, preferably such that it is not movable from a fixed and desired position in the cage by an animal. 902 is a surface on which an animal may be, such that the animal may receive a stimulus and respond. The animal may also be weighed while on this surface. 908 is a top plate supporting surface 902. 908 may be a circuit board (PCB), The top surface 902 should be sterilizable and non-chewable by an animal. 903 are transceivers to communicate with the first apparatus, here aimed upward and located on, in or under the top plate. If under the top plate, at least a portion of the top plate 902 needs to be transparent to the transceiver signal. As shown, transceivers 903 are redundant and each has two elements, one for transmitting, such as an LED and one for receiving, such as a photo or light sensor. Elements 904, which may either a single element or multiple elements, provide both electrical connection and mechanical connection from the top plate 908. 907 is a motion communication plate that acts as an intermediary element between the top plate 908 and sensor and electronics within the main body of the apparatus, 901. This intermediate element may be for mechanical connection, electronic connection or both. In one embodiment this element 907 is a flexible membrane secured on the perimeter of the second apparatus shell through which both mechanical and electronics penetrate via element or elements 904. Such a flexible membrane provides the body of the second apparatus, 901 with a seal against pathogens. Ideally, the top portion comprising predominantly top plate 908 and its elements is readily separable from the second apparatus body 901 so that each may be sterilized separately. A power source within the body 901 (such as shown as element 36 in FIG. 3) is ideally charged via connector(s) 904 while the second apparatus is so disassembled.

909 is a motion sensor, such as discussed elsewhere herein, to detect the animal response. It is mechanically coupled directly or indirectly to the surface 902. It may be on, in or under the top plate 908, or in the body of the second apparatus 901, as shown. Electronics within the body of the second apparatus, 901, are not shown. Typically such electronics comprise a power source such as a battery, a processor, and non-transitive memory to hold both code and data, and interfaces to the communication transceivers 903.

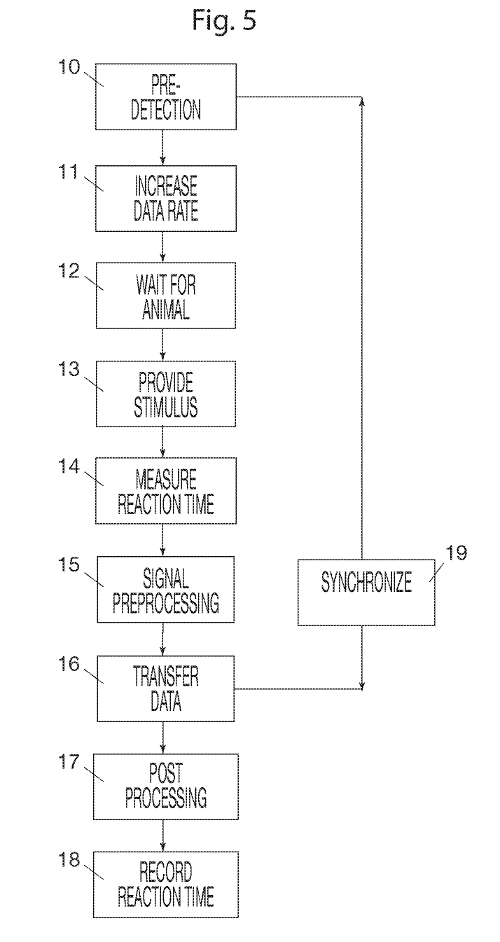

Turning now to FIG. 5 we see a flowchart of embodiments. Predetection step 10 is an optional step to enable the second apparatus to perform steps 11 through 16 and 19 effectively. It may increase power or data rate or both, for example. Such predetection may use a load cell, capacitive sensor, optical sensor, or other sensor to determine that at a portion of an animal is on the top surface of the second apparatus. Alternatively, the second apparatus may receive predetection signal from the first apparatus. One may think, very generally, as this step “waking up” the second apparatus to effectively perform steps of embodiments.

Step 11 is an optional step for the second apparatus to increase data rate in communicating with the first apparatus. Two different data rates are convenient for conserving power consumption in the second apparatus between operations, such as when no animal is on the apparatus and there is no pending data to transmit or receive. Such dual data-rates are specifically claimed as embodiments.

Step 12 waits for the animal to be appropriately positioned on the apparatus. For example, fully on. Also, this step may comprise waiting for a command from the first apparatus. In such a case, this step may be a long wait.

Step 13 provides a stimulus to an animal. This stimulus is discussed elsewhere herein. The stimulus may come from either the first or the second apparatus, or from some other source.

In step 14, the second apparatus measures the animal's reaction time from the stimulus, as is discussed elsewhere herein.

Step 15 is an optional preprocessing step, as discussed elsewhere herein. If not performed, then raw or nearly raw sensor data is transferred to the first apparatus in step 16. Such preprocessing may include averaging, smoothing, amplitude detection, zeroing, linearization, bounding, baseline subtraction, outlier elimination, peak detection, integration or differentiation, hysteresis removal, and the like. An animal's reaction may be detected as a distance translation, motion, velocity or acceleration, in one or more axes, or in any combination. An accelerometer is a good detector; acceleration in multiple axes may be added, multiplied, used as a filter, or otherwise combined. The goal of any such preprocessing is to provide consistent measurements for consistent reaction times. For example, multiple reaction times of each of a plurality of similar animals could be measured, and then reprocessing algorithms and coefficients applied to raw sensor data selected to minimize a standard deviation of such measurements. Preprocessing also minimizes the amount of data transmitted in step 16.

In step 16 reaction time data is transmitted from the second apparatus to the first apparatus. Raw data from one or more sensors is likely to be the largest amount of data, while reduction via preprocessing to a single scalar, such 25 ms, or a deviation from an expected scalar, such as +4 ms, is likely the most minimal amount of data. Some preprocessing, as discussed elsewhere herein, likely produces an intermediate amount of data. This option provides for more complex processing outside of the second apparatus. Reaction times may be in the range of 5 to 100 ms, 10 to 80 ms, 15 to 70 ms, or another range.

Step 19 is time synchronization between the first and second apparatuses. This is necessary if the stimulus is from the first apparatus. One such time synchronization is the use of precision clocks, including GPS. Another such time synchronization is for the second apparatus to have a sensor that directly detects the stimulus. A third such time synchronization is to use one more communication packets to explicitly or implicitly include a time. Explicitly incudes putting a time stamp field in a packet. Implicitly includes timing having a known reference point in a packet, such as its starting time, be a time mark. For example, the second apparatus could measure reaction time relative a past packet sent or received. In this way, the first apparatus could provide the necessary offset and thus correction to compute arithmetically the actual response time. An alternative embodiment is to provide time synchronization after the response time data is sent in step 16. In this case, the first apparatus or some other apparatus applies the necessary time correction in step 17. Thus, such time synchronization, step 19, may be performed at any time from prior to step 10 to prior to step 18. A preferred embodiment is to perform the synchronization 19 some time between, inclusively, steps 10 to 16, as shown in the Figure.

Step 17 is an optional post-processing step. Such a step may perform any of or more than the preprocessing operations described elsewhere herein. This step may also provide validation, time and date stamping, and association with an animal, cage or study. This step may be performed in the first apparatus or elsewhere. This step may provide information to people or a user interface (UI). This step may comprise communicating reaction time data to another device or location.

Step 18 records a proper animal reaction time in non-transitive memory, such as a database. Steps 17 and 18 may be combined or performed in either order.

Not shown in FIG. 5 is an enablement or command to perform any or all steps of embodiments by the second apparatus. Such an enablement or command may be implicit, may be includes in some other command or configuration, or may be a decision made by the logic in the second apparatus, or may be a signal provided by the first apparatus to the second apparatus. It is desirable to control the number and timing of animal stimuli, such as by the use of such an enablement or command. Surprising an animal too frequently stresses the animal, may cause habituation, and may alter or invalidate the results of a study. Providing a stimulus one per day may be appropriate for tracking animal health, disease, prevention or recovery. Stimulus more frequently, such as every ¼, ⅓, ½, 1, 2, 3, 4, 6 or 12 hours may be appropriate for determining effects of a particular treatment or medication. Such frequent stimulus may also be used to measure habituation or dishabituation.

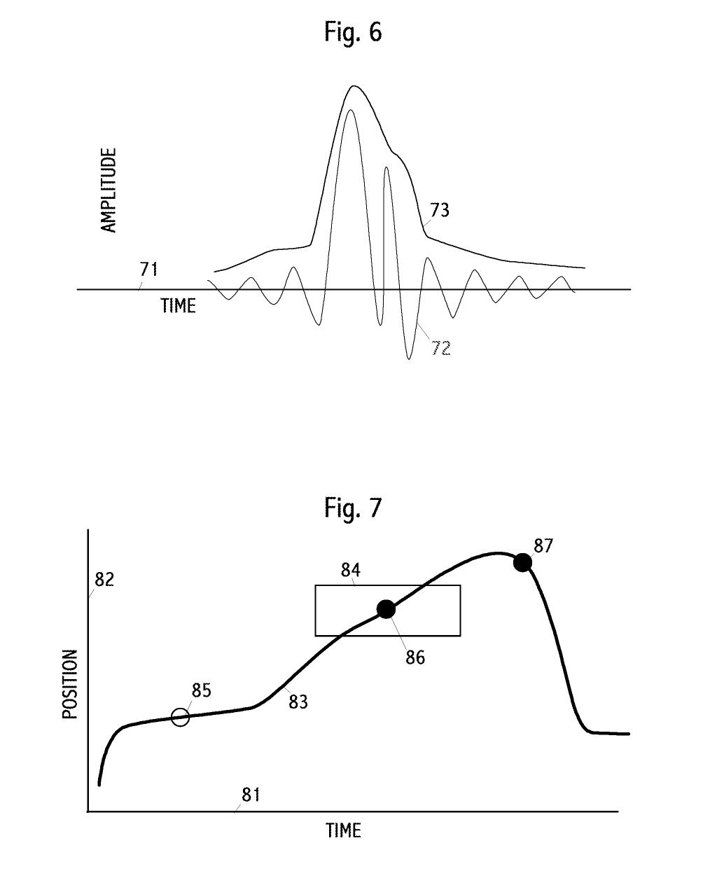

Turning now to FIG. 6, we see schematic renderings of exemplary raw sensor data and preprocessed data, as discussed elsewhere herein. 71 is a time line and also may be viewed as a zero or offset baseline. 72 shows possible raw sensor data. This data may show resonance of some portion of the second apparatus, as visible in the Figure. 73 shows a possible result of preprocessing of the signal 72. Here, in this example, amplitude detection, baseline offset, and smoothing (averaging or frequency filtering, for example) are shown. The vertical axis in this Figure is arbitrary amplitude of one or more appropriate units of measure, with no scale.

Turning now to FIG. 7 we see three possible places in time where animal ID may be performed, for cages that have more than one animal, multi-housed. The vertical axis 82 a schematic for animal position. Only one schematic axis is shown, even though actual animal position in a cage is likely more or many more axes, including for example, a vertical axis and animal shape or animal activity. The horizontal axis is time with no scale. Box 84 represents schematically both location and time for an animal on the second apparatus. Dots 85, 84, and 87 represent one or more locations and time when an animal may have its ID detected. Typically, the only animal ID necessary is to distinguish one animal in a cage from all other animals in the same cage. However, animal ID may comprise more information. Dot 85 shows detecting animal ID prior to the animal being tested for reaction time. Dot 86 shows detecting an animal ID while the animal is the second apparatus. Dot 87 shows detecting an animal ID after the animal is tested for reaction time. Animal ID may be a tail marking, RFID, or other animal marking, such as ear cuts or footpad tattoos. Animal ID may be detected by a camera from above, a camera below, or an RFID reader.

Embodiments include tremor detection and data transmission to the first apparatus, with or without reaction time measurements. Mouse tremors may be in the range of 11 to 14 Hz or 8 to 20 Hz. The accelerometer in the second apparatus may be used for this embodiment. An animal may or may not be stressed. The stimulus as described and claimed may be replaced with a “stressor,” which may be a loud noise, change in lighting, change in temperature, electrical shock, olfactory stimulation, or change to husbandry provisions. As for other embodiments, detection of the animal is necessary, the animal need to be fully (or nearly fully, or more than 50% by weight) on the apparatus. Detection and communication steps are the same or similar. Detecting a delay from the stressor to the tremor may or may not be used, depending on embodiments. Measured amplitude of tremor is required in one embodiment. Measured frequency or frequency spectrum is required in one embodiment. Optional preprocessing, post-processing, animal ID, apparatus limitations, and recording are as described for other embodiments.

Embodiments are claimed wherein the second apparatus performs both reaction time measurement steps and tremor detection steps.

Embodiments are claimed wherein the second apparatus performs both animal weight measurement and transmission, and tremor detection steps.

Embodiments are claimed wherein the second apparatus performs reaction time measurement steps, animal weight measurement and transmission, and tremor detection steps.

### Notes on Claims

Notes below are on claims as numbered in the original filing. Numbers below may not correspond to claim numbers as issued. Material below may be used for claim construction if such construction is necessary to establish or maintain a valid claim. Otherwise, material below reflects embodiments only and in no way limits claim scope.

Regarding original claim 1, “communicating a command” is broad in scope. As discussed elsewhere herein, such communication may implied, part of another command, provided with device software, configuration or installation. An alternative embodiment is claimed wherein step (a) is replaced with: “communicating a command to a second apparatus. “Fully on the apparatus” means most of the weight of the animal or at least two out of four feet, such a response to a stimulus may be detected reliably. An animal tail need not be on the apparatus. The animal may be standing on four feet, three feet, or standing up on two feet. The animal may be still or moving around. “Measuring a reaction time” in step (d) means recording data from one or more reaction time sensors and recording a time of that data. Determining a time between the stimulus and the time of the sensor data collection may not be a final reaction time, as correction for time-base offsets may be required. The “transmitting” in step (e) refers to either uncorrected time from the measuring in step (d) or corrected time, if any correction of offset is required. “Sterilizable” refers to the effective elimination of pathogens such that a study result involving the cage and animal is valid. “Wireless” means the device communicates via at least one transmitter using optical or radio communication. In an alternative embodiment, step (e) is replaced with, “transmitting the reaction

Regarding original claim 2, the “detecting” may be performed by the second apparatus, the first apparatus, or by another apparatus, or by any combination. The detecting may be performed by the first apparatus and communicated to the second apparatus. The “detecting” in this claim does not require an animal to be fully on the second apparatus. It might be partially on. In an alternative embodiment, the step is replaced by, “(f) detecting an animal on the second apparatus; wherein step (f) occurs prior to step (c).”

Regarding claim 4, a “time synchronization signal” is discussed elsewhere herein. This is to be broadly construed, as there are many methods and times for the first and second apparatuses to time synchronize. The necessary precision of the time synchronization is such that the embodiment performs its intended function. That is, final response times recorded must be within the precision requirements of a study. Such precision might be within 0.1, 0.25, 0.5, 1, 2, 3, 5, 10, 25, 50 milliseconds (ms), or another range.

Regarding claims 5 and 6s, time synchronization may be fully or in part the transmitting of a stimulus from the first apparatus and receiving the stimulus by the second apparatus. No other synchronization signal may be necessary.

Regarding claim 7, as discusses elsewhere herein, many different sounds, lengths and volume levels may be appropriate in an application. One sound is a white noise burst for 50 ms at 100 db.

Regarding claims 10 and 11, a “home cage” is a term of the art. Intent of application of some embodiments is that an animal response time may be measured with no human handling of the animal.

Regarding claim 12, this embodiment refers to the use of a dedicated first apparatus for each cage.

Regarding claim 13, alternative embodiments use other animal size metrics, such as “maximum diameter of the animal” or an average or maximum length of the animal, excluding a tail.

Regarding step 14, a “disease model” should be construed broadly.

Regarding step 15, alternative embodiments replace “behavior model” with “disease model” or “treatment model.”

### DEFINITIONS

“Pathogen-free”—means the population of microbes, including but not limited to bacteria, viruses, prions and toxins, relevant to an experiment, test or study (“study”), is sufficiently reduced to meet the needs of the study, or to impact or alter study results, or to alter the credibility or repeatability of study results, for studies using the vivarium, and to not impact the health, performance or behavior of the target animal population in the vivarium or of the workers.

“Sterile”—sufficiently pathogen-free to meet the needs of a study.

“Subset”—May include any non-zero number of elements from a set, including all elements from the set.

Ideal, Ideally, Optimum and Preferred—Use of the words, “ideal,” “ideally,” “optimum,” “optimum,” “should” and “preferred,” when used in the context of describing this invention, refer specifically a best mode for one or more embodiments for one or more applications of this invention. Such best modes are non-limiting, and may not be the best mode for all embodiments, applications, or implementation technologies, as one trained in the art will appreciate.

All examples are sample embodiments. In particular, the phrase “invention” should be interpreted under all conditions to mean, “an embodiment of this invention.” Examples, scenarios, and drawings are non-limiting. The only limitations of this invention are in the claims.

May, Could, Option, Mode, Alternative and Feature—Use of the words, “may,” “could,” “option,” “optional,” “mode,” “alternative,” “typical,” “ideal,” and “feature,” when used in the context of describing this invention, refer specifically to various embodiments of this invention. Described benefits refer only to those embodiments that provide that benefit. All descriptions herein are non-limiting, as one trained in the art appreciates.

Embodiments of this invention explicitly include all combinations and sub-combinations of all features, elements and limitation of all claims. Embodiments of this invention explicitly include all combinations and sub-combinations of all features, elements, examples, embodiments, tables, values, ranges, and drawings in the specification and drawings. Embodiments of this invention explicitly include devices and systems to implement any combination of all methods described in the claims, specification and drawings. Embodiments of the methods of invention explicitly include all combinations of dependent method claim steps, in any functional order. Embodiments of the methods of invention explicitly include, when referencing any device claim, a substation thereof to any and all other device claims, including all combinations of elements in device claims. Claims for devices and systems may be restricted to perform only the methods of embodiments or claims.

We claim:

1. A method of measuring a reaction time of an animal in a cage comprising the steps of:
(a) communicating a command from a first apparatus to a second apparatus;
(b) waiting for the animal to be fully on the second apparatus;
(c) providing a stimulus to the animal;
(d) measuring the reaction time of the animal using the second apparatus;
(e) transmitting the reaction time from the second apparatus to the first apparatus;
wherein the second apparatus is a sterilizable wireless device in the cage.

2. The method of claim 1 further comprising the additional step:
(f) detecting an animal on the second apparatus;
wherein step (f) occurs prior to step (b).

3. The method of claim 2 further comprising the additional step:
(g) sending a signal from the second apparatus to the first apparatus responsive to the detecting in step (f);
wherein step (g) occurs after step (f) and prior to step (c).

4. The method of claim 1 further comprising the additional step:
(h) communicating a time synchronization signal between the first apparatus and the second apparatus;
wherein step (h) occurs at any time from prior to step(a) to after step (e).

5. The method of claim 4:
wherein at least a portion of the communicating in step (h) comprises the providing the stimulus in step (c).

6. The method of claim 1:
wherein the communicating in step (h) consists of the providing the stimulus in step (c).

7. The method of claim 1:
wherein the communicating in step (h) consists of the providing the stimulus in step (c); and
wherein the stimulus is a sound.

8. The method of claim 1:
wherein the communicating in step (h) consists of the providing the stimulus in step (c); and
wherein the stimulus is a light.

9. The method of claim 1 further comprising additional the step:
(i) preprocessing a reaction motion from the animal;
wherein the preprocessing in step (i) occurs after step (c) and prior to step (e).

10. The method of claim 1:
wherein the cage is a home cage of the animal.

11. The method of claim 1:
wherein steps (a) through (e) are performed automatically.

12. The method of claim 1:
wherein the second apparatus is a sterilizable, wireless device in the cage and the first apparatus performs step (a) for no other cage that the cage.

13. The method of claim 1:
wherein the second apparatus comprises two or more redundant optical communication devices on a top of the apparatus with a distance separating at least two of the redundant optical communication devices larger than an average diameter of the animal.

14. The method of claim 1 further comprising the steps:
(j) repeating steps (a) through (e) sequentially for two or more days;
(k) aggregating the animal reaction times of the repeating into a sequence;
(l) comparing the sequence statistically to a first disease model;
(m) generating a disease likelihood metric responsive to the comparing in step (l).

15. The method of claim 1 further comprising the steps:
(n) repeating steps (a) through (e) sequentially;
(o) aggregating the animal reaction times of the repeating into a sequence;
(p) comparing the sequence statistically to a first behavior model;
(q) generating a behavior metric responsive to the comparing in step (p);
wherein the behavior model is habituation.

16. The method of claim 1:
wherein the second apparatus comprises a weight scale.

17. The method of claim 1:
wherein the second apparatus comprises a first communication rate and a second communication rate;
wherein the first communicate is used when the second apparatus is not currently executing an animal related function and the second communication rate is used when the second apparatus is performing an animal related function; and
wherein the selection of the communication rate is selected solely by the second apparatus.

18. The method of claim 1:
wherein step (c) is performed during the animal's nighttime.

19. The method of claim 1:
wherein the waiting in step (b) is performed by the second apparatus.

20. The method of claim 1:
wherein the waiting in step (b) is performed by the first apparatus.

21. The method of claim 1:
wherein the cage houses one or more animals in addition to the animal.

22. The method of claim 1 comprising the additional step:
(r) detecting a cage-unique animal ID of the animal;
wherein the detecting in step (r) occurs prior to step (c);
wherein step (c) is responsive to step (r); and
wherein the cage houses one or more animals in addition to the animal.

23. The method of claim 1:
wherein the measuring in step (d) is responsive to an acceleration of a top of the second apparatus.

24. The method of claim 1 comprising the additional step:
wherein the waiting in step (b) is responsive to a weight of the animal on a top of the second apparatus.

25. A device that implements the method of claim 1.

26. A vivarium that implements the method of claim 1.
